(19) United States
(12) Patent Application Publication
(10) Pub. No.: US 2026/0218275 A1
(43) Pub. Date: Jul. 30, 2026

(54) Composition for detecting target nucleic acid and method for detecting target nucleic acid using the same

(71) Applicant: Hoseo University Academic Cooperation Foundation, Asan-si (KR)

(72) Inventors: Kue Whan JANG, Asan-si (KR); Sung Soo NA, Seoul (KR); June Seok YOU, Gumi-si (KR)

(21) Appl. No.: 19/434,112

(22) Filed: Dec. 29, 2025

(30) Foreign Application Priority Data
Dec. 27, 2024 (KR) 10-2024-0199303

(51) Int. Cl. C12Q 1/6825; G01N 21/64

(52) U.S. Cl. CPC C12Q 1/6825; G01N 21/6428; C12Q 2600/178; G01N 2021/6432

(57) ABSTRACT

The present invention relates to a DNA composition for detecting a target nucleic acid and a method for detecting a target nucleic acid using the same, and more particularly, to a composition including hairpin DNA for detecting a target nucleic acid and to a method for detecting a target nucleic acid using the composition. According to the present invention, there is provided a hairpin DNA composition having nucleotide sequences designed to selectively react only with a specific target nucleic acid such as microRNA-569, so that detection of the target nucleic acid proceeds through emission of a fluorescence signal according to a structural change of the hairpin DNA. Accordingly, even when a small amount of the target nucleic acid is present, DNA structures are continuously generated to amplify the reaction, thereby making it possible to detect the target nucleic acid most effectively.

### BACKGROUND OF THE INVENTION

### Field of the Invention

The present invention relates to a DNA composition for detecting a target nucleic acid and a method for detecting a target nucleic acid using the same, and more particularly, to a composition including hairpin DNA for detecting a target nucleic acid and to a method for detecting a target nucleic acid using the composition.

### Description of the Related Art

Among target nucleic acids, microRNA-569 nucleic acid is a type of microRNA and is known in particular to inhibit tumor metastasis of pancreatic cancer. It has also been demonstrated that targeting microRNA-569 may potentially be helpful for patients with ovarian cancer or breast cancer. Furthermore, the role of microRNA-569 nucleic acid observed in recent studies of lung cancer cells suggests that it may serve as a novel and promising therapeutic target and as a new biomarker for detecting lung cancer.

Accordingly, it can be seen that detecting microRNA-569 nucleic acid may play an important role in the diagnosis and treatment of pancreatic cancer and lung cancer as described above.

However, to date, no technique capable of effectively detecting microRNA-569 nucleic acid has been presented.

### PRIOR ART LITERATURE

### Patent Literature

(Patent Literature 1) [1] KR 10-2022-0069865 A

### SUMMARY OF THE INVENTION

The present invention has been conceived to solve the above-described problems, and an object of the present invention is to provide a configuration of a DNA composition for most effectively detecting a target nucleic acid, particularly microRNA-569 nucleic acid, and a method for detecting a target nucleic acid using the same.

To achieve the above object, a DNA composition for detecting a target nucleic acid according to the present invention comprises: a single-stranded hairpin DNA1 comprising a nucleotide sequence capable of complementary binding to a target nucleic acid, and having a stem portion in which portions of opposite ends are complementarily bound to each other and an unhybridized loop portion in the form of a loop; a single-stranded hairpin DNA2 comprising a nucleotide sequence capable of complementary binding to the hairpin DNA1, and having a stem portion in which portions of opposite ends are complementarily bound to each other and an unhybridized loop portion in the form of a loop; and a hairpin DNA3 comprising a nucleotide sequence capable of complementary binding to the hairpin DNA2, and having a stem portion in which portions of opposite ends are complementarily bound to each other and an unhybridized loop portion in the form of a loop, the hairpin DNA3 having a fluorophore at one end and a quencher, which is a fluorescence quencher, at the opposite end.

The hairpin DNA1 hybridizes with the target nucleic acid so that the stem portion of the hairpin DNA1 is opened. Subsequently, the hairpin DNA2 hybridizes with the hairpin DNA1 so that the stem portion of the hairpin DNA2 is opened. Subsequently, the hairpin DNA3 hybridizes with the hairpin DNA2 so that the stem portion of the hairpin DNA3 is opened, and another portion of the hairpin DNA3 can displace the target nucleic acid bound to the hairpin DNA1 and hybridizes with the hairpin DNA1 at that portion to form a Y-shaped DNA structure.

In the Y-shaped DNA structure, a fluorescence signal may be emitted from the fluorophore provided at one end of the opened hairpin DNA3, whereby detection of the target nucleic acid may be achieved.

The target nucleic acid may be microRNA-569 having the nucleotide sequence of SEQ ID NO: 1 (hereinafter, referred to as "miRNA-569").

The hairpin DNA1 may have the nucleotide sequence of SEQ ID NO: 2, the hairpin DNA2 may have the nucleotide sequence of SEQ ID NO: 3, and the hairpin DNA3 may have the nucleotide sequence of SEQ ID NO: 4.

The hairpin DNA1 may have the nucleotide sequence of SEQ ID NO: 5, the hairpin DNA2 may have the nucleotide sequence of SEQ ID NO: 6, and the hairpin DNA3 may have the nucleotide sequence of SEQ ID NO: 7.

According to another aspect of the present invention, there is disclosed a target nucleic acid detection sensor comprising the DNA composition for detecting a target nucleic acid.

According to still another aspect of the present invention, there is disclosed a method for detecting a target nucleic acid using the DNA composition for detecting a target nucleic acid.

The method for detecting a target nucleic acid comprises: (a) complementarily binding the hairpin DNA1 to the target nucleic acid so that the stem portion of the hairpin DNA1 is opened; (b) complementarily binding the hairpin DNA2 to the hairpin DNA1 so that the stem portion of the hairpin DNA2 is opened; (c) complementarily binding the hairpin DNA3 to the hairpin DNA2 so that the stem portion of the hairpin DNA3 is opened; and (d) displacing, by another portion of the hairpin DNA3, the target nucleic acid bound to the hairpin DNA1 and hybridizing with the hairpin DNA1 at that portion to form a Y-shaped DNA structure.

In the Y-shaped DNA structure, a fluorescence signal may be emitted from the fluorophore provided at one end of the opened hairpin DNA3, whereby detection of the target nucleic acid may be achieved.

The target nucleic acid may be microRNA-569 having the nucleotide sequence of SEQ ID NO: 1 (hereinafter, referred to as "miRNA-569").

The hairpin DNA1 may have the nucleotide sequence of SEQ ID NO: 2, the hairpin DNA2 may have the nucleotide sequence of SEQ ID NO: 3, and the hairpin DNA3 may have the nucleotide sequence of SEQ ID NO: 4.

The hairpin DNA1 may have the nucleotide sequence of SEQ ID NO: 5, the hairpin DNA2 may have the nucleotide sequence of SEQ ID NO: 6, and the hairpin DNA3 may have the nucleotide sequence of SEQ ID NO: 7.

According to the present invention, there is provided a hairpin DNA composition having nucleotide sequences designed to selectively react only with 0 a specific target nucleic acid such as microRNA-569, so that detection of the target nucleic acid proceeds through emission of a fluorescence signal according to a structural change of the hairpin DNA. Accordingly, even when a small amount of the target nucleic acid is present, DNA structures are continuously generated to amplify the reaction, thereby making it possible to detect the target nucleic acid most effectively.

### BRIEF DESCRIPTION OF THE DRAWINGS

FIG. 1 is a view illustrating the configuration of the DNA composition of the present invention for detecting a target nucleic acid.

FIG. 2 is a view illustrating a process of detecting a target nucleic acid using the DNA composition of the present invention.

FIG. 3 is a view illustrating results of detecting microRNA-569 nucleic acid using a hairpin DNA composition having nucleotide sequences according to a first embodiment.

FIG. 4 is a view illustrating results of detecting microRNA-569 nucleic acid using a hairpin DNA composition having nucleotide sequences according to a second embodiment.

### DETAILED DESCRIPTION OF THE INVENTION

Hereinafter, preferred embodiments of the present invention will be described in detail with reference to the accompanying drawings. Prior to this, it should be understood that the terms and words used in the present specification and claims should not be construed as being limited to ordinary or dictionary meanings, but should be construed as having meanings and concepts consistent with the technical spirit of the present invention, based on the principle that the inventor may appropriately define the concepts of terms in order to describe his or her invention in the best manner. Therefore, the embodiments described in the present specification and the configurations shown in the drawings are merely the most preferred embodiments of the present invention and do not represent all of the technical spirit of the present invention, and it should be understood that various equivalents and modifications capable of replacing them may be made at the time of the filing of the present application.

Hereinafter, a method for detecting a specific target nucleic acid using a specific DNA composition will be described, with particularfocus on, among target nucleic acids, a method for detecting microRNA-569 as one embodiment, and a DNA composition for use in such detection.

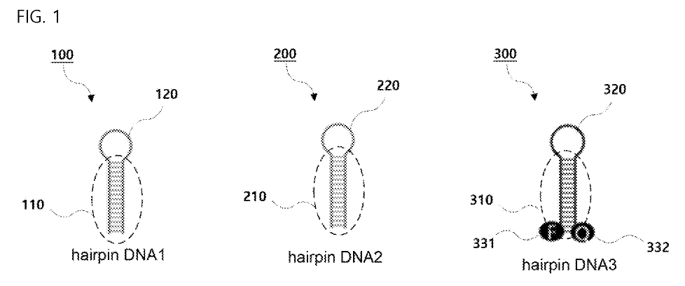

FIG. 1 is a view illustrating the configuration of the DNA composition of the present invention for detecting a target nucleic acid. The nucleotide sequences of the RNAs and DNAs used in the present invention are as follows:

SEQ ID NO: 1: microRNA-569

5'-AGUUAAUGAAUCCUGGAAAGU-3'

SEQ ID NO: 2: hairpin DNA1

5'-

TAGAAACTTTCCAGGATTCATTAACTTCTAGCAAGGTTAATGAATCCTGG-3'

SEQ ID NO: 3: hairpin DNA2

5'-

CCAGGATTCATTAACCTTGCTAGAACCTGGAAAGTTTCTAGCAAGGTTAA-3'

SEQ ID NO: 4: hairpin DNA3

5'-

TTAACCTTGCTAGAAACTTTCCAGGGTTAATGAATCCTGGAAAGTTTCTA-3'

SEQ ID NO: 5: hairpin DNA1

5'- AAAAAAAAAATAGAAACTTTCCAGGATTCATTAACTTCTAGCAAGGTTAATG AATCCTGG-3'

SEQ ID NO: 6: hairpin DNA2

5'-

AAAAAAAAAACCAGGATTCATTAACCTTGCTAGAACCTGGAAAGTTTCTAGCAAGGTTAA-3'

SEQ ID NO: 7: hairpin DNA3

5'-

AAAAAAAAAATTAACCTTGCTAGAAACTTTCCAGGGTTAATGAATCCTGGAAAGTTTCTA-3'

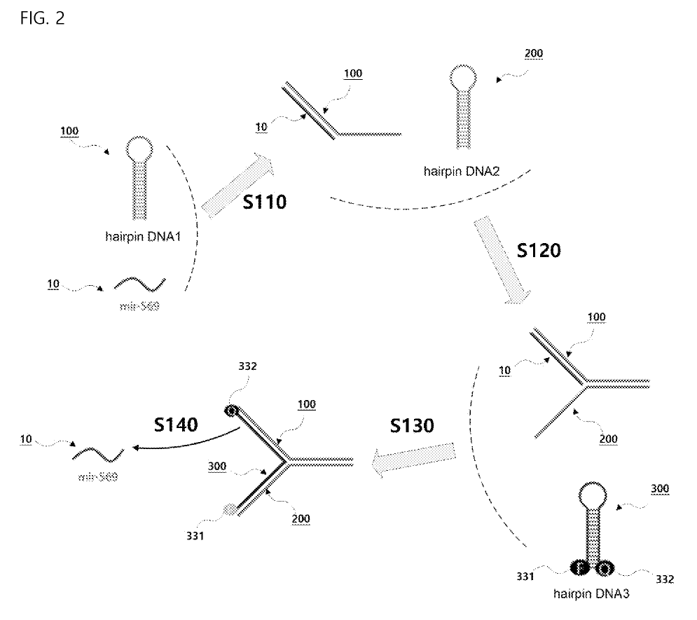

As one embodiment, the target nucleic acid to be detected in the present invention is microRNA-569 (hereinafter, referred to as "miRNA-569") (10; see FIGS. 1 and 2). miRNA-569 (10) has the nucleotide sequence of SEQ ID NO: 1. As described above, it is a nucleic acid that functions as a biomarker for diagnosis of pancreatic cancer, lung cancer, and the like.

The DNA composition of the present invention for detecting miRNA-569 (10) includes hairpin DNA1 (100; see FIGS. 1 and 2), hairpin DNA2 (200; see FIGS. 1 and 2), and hairpin DNA3 (300; see FIGS. 1 and 2). Each of hairpin DNA1 (100), hairpin DNA2 (200), and hairpin DNA3 (300) is composed of single- stranded DNA. Each hairpin DNA will be described below with reference to FIG. 1.

### 1) Hairpin DNA composition according to a first embodiment

The hairpin DNA composition according to the first embodiment includes the above-described hairpin DNA1 (100) of SEQ ID NO: 2, hairpin DNA2 (200) of SEQ ID NO: 3, and hairpin DNA3 (300) of SEQ ID NO: 4.

As shown in FIG. 1, each of hairpin DNA1 (100), hairpin DNA2 (200), and hairpin DNA3 (300) of SEQ ID NOS: 2, 3, and 4 has a stem portion (110, 210, 310), which is a portion complementarily bound to each other, and a loop portion (120, 220, 320), which is aloop-shaped portion that is not complementarily bound.

In particular, a fluorophore (F, 331) that generates fluorescence energy is attached to one end of hairpin DNA3 (300), and a quencher (Q, 332), which is a fluorescence quencher, is attached to the opposite end thereof. When the fluorophore (F, 331) and the quencher (Q, 332) are located in close proximity to each other as shown in FIG. 1, the quencher (Q, 332) absorbs the fluorescence energy generated from the fluorophore (F, 331), thereby preventing a fluorescence signal from being emitted to the outside.

### 2) Hairpin DNA composition according to a second embodiment

The hairpin DNA composition according to the second embodiment includes the above-described hairpin DNA1 (100) of SEQ ID NO: 5, hairpin DNA2 (200) of SEQ ID NO: 6, and hairpin DNA3 (300) of SEQ ID NO: 7.

As shown in FIG. 1, each of hairpin DNA1 (100), hairpin DNA2 (200), and hairpin DNA3 (300) of SEQ ID NOS: 5, 6, and 7 has a stem portion (110, 210, 310), which is a portion complementarily bound to each other, and a loop portion (120, 220, 320), which is aloop-shaped portion that is not complementarily bound.

Hairpin DNA1, hairpin DNA2, and hairpin DNA3 of SEQ ID NOS: 5, 6, and 7 each include the same nucleotide sequence as hairpin DNA1, hairpin DNA2, and hairpin DNA3 of SEQ ID NOS: 2, 3, and 4, respectively, and further each have ten additional A residues connected in front of the same nucleotide sequence.

As in the first embodiment, a fluorophore (F, 331) that generates fluorescence energy is attached to one end of hairpin DNA3 (300), and a quencher (Q, 332) is attached to the opposite end thereof. When the fluorophore (F, 331) and the quencher (Q, 332) are located in close proximity to each other as shown in FIG. 1, the quencher (Q, 332) absorbs the fluorescence energy generated from the fluorophore (F, 331), thereby preventing a fluorescence signal from being emitted to the outside.

FIG. 2 is a view illustrating a process of detecting a target nucleic acid using the DNA composition of the present invention described with reference to FIG. 1.

Hairpin DNA1 (100) includes a sequence that hybridizes with miRNA-569 (10), which is the target nucleic acid. When miRNA-569 (10) and hairpin DNA1 (100) hybridizes with each other at this sequence, the stem portion (110) of hairpin DNA1 (100) is opened by the driving force thereof (S110).

At this time, the 'ACTTTCCAGGATTCATTAAC' region of hairpin DNA1 (100) may hybridize with miRNA-569 (10).

Subsequently, hairpin DNA2 (200), which is capable of complementarily binding to a portion of the opened hairpin DNA1 (100), hybridizes with hairpin DNA1 (100), whereby the stem portion (210) of hairpin DNA2 (200) is opened (S 120).

At this time, the 'CCAGGATTCATTAACCTTGCTAGAA' region of hairpin DNA2 (200) may hybridize with the 'TTCTAGCAAGGTTAATGAATCCTGG' region of hairpin DNA1 (100).

Thereafter, hairpin DNA3 (300), which is capable of complementarily binding to a portion of the opened hairpin DNA2 (200), hybridizes with hairpin DNA2 (200), whereby the stem portion (310) of hairpin DNA3 (300) is opened, and also hybridizes with hairpin DNA1 (100) at another region to generate a Y- shaped DNA structure (S130). At this time, the opened hairpin DNA3 (300), while hybridizing with hairpin DNA1 (100), displaces miRNA-569 (10), which had been complementarily bound to hairpin DNA1 (100) at that region (S140).

At this time, the 'TTAACCTTGCTAGAAACTTTCCAGG' region of hairpin DNA3 (300) may hybridize with the 'CCTGGAAAGTTTCTAGCAAGGTTAA' region of hairpin DNA2 (200), and the 'GTTAATGAATCCTGGAAAGTTTCTA' region of hairpin DNA3 (300) may hybridize with the 'TAGAAACTTTCCAGGATTCATTAAC' egion of hairpin DNA1 (100).

Before hairpin DNA3 (300) is opened, the fluorophore (F, 331) attached to one end of the stem portion (310) is located in close proximity to the quencher (Q, 332) attached to the opposite end. Accordingly, the fluorescence energy generated from the fluorophore (F, 331) is transferred to the quencher (Q, 332), which is a fluorescence quencher, and thus is not emitted to the outside as a fluorescence signal but remains in a quenched state by the quencher (Q, 332).

However, when hairpin DNA3 (300) hybridizes with hairpin DNA2 (200) and is opened to form the Y-shaped DNA structure (S130), the fluorophore (F, 331) and the quencher (Q, 332) of hairpin DNA3 (300) are sufficiently separated from each other, so that the fluorescence signal of the fluorophore (F, 331) is emitted to the outside.

Furthermore, miRNA-569 (10), which has been displaced from hairpin DNA1 (100) by the opened hairpin DNA3 (300), reacts again with another hairpin DNA1 and repeats the same process described above while repeatedly generating the Y-shaped DNA structure, thereby amplifying generation of the Y- shaped DNA structure.

In this manner, even when a small amount of miRNA-569 (10) is present, the fluorescence signal emitted while continuously amplifying the generation of 0 the Y-shaped DNA structure is further amplified, whereby detection of miRNA-569 (10) is carried out.

The hairpin DNA1 (100), hairpin DNA2 (200), and hairpin DNA3 (300) used in the above-described detection process of the target nucleic acid miRNA- 569 (10) may be DNAs corresponding to SEQ ID NOS: 2, 3, and 4, respectively, as a first embodiment, or may be DNAs corresponding to SEQ ID NOS: 5, 6, and 7, respectively, as a second embodiment.

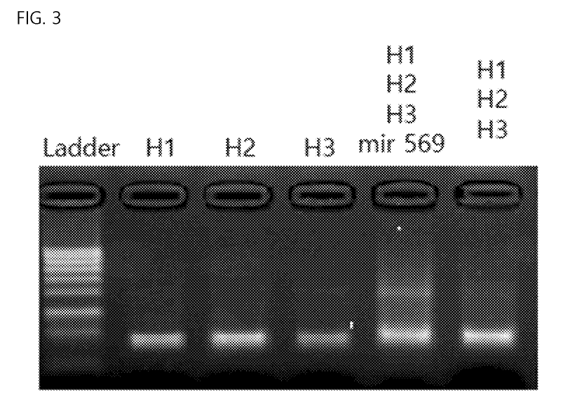

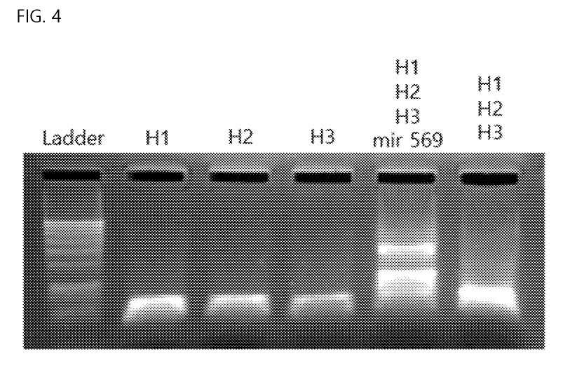

Results of detection of the target nucleic acid miRNA-569 (10) obtained using the DNAs respectively corresponding to SEQ ID NOS: 2, 3, and 4 as the first embodiment are shown in FIG. 3, and results of detection of the target nucleic acid miRNA-569 (10) obtained using the DNAs respectively corresponding to SEQ ID NOS: 5, 6, and 7 as the second embodiment are shown in FIG. 4.

A composition including hairpin DNA1 (100), hairpin DNA2 (200), and hairpin DNA3 (300) as described above may be used in a sensor for detecting the target nucleic acid miRNA-569.

Furthermore, with respect to a specific microRNA, which is a target nucleic acid other than miRNA-569, a composition of hairpin DNA1, hairpin DNA2, and hairpin DNA3 that forms a Y-shaped DNA structure through sequential complementary binding as described above may also be included in the present invention. In this case, hairpin DNA1, hairpin DNA2, and hairpin DNA3 should have nucleotide sequences different from SEQ ID NOS: 2 to 7 and capable of sequential complementary binding with the corresponding microRNA. Also in this case, a fluorophore should be attached to one end of hairpin DNA3, and a quencher, which is a fluorescence quencher, should be attached to the opposite end thereof.

FIG. 3 is a view illustrating results of detecting miRNA-569 nucleic acid using a hairpin DNA composition having nucleotide sequences according to a first embodiment, and FIG. 4 is a view illustrating results of detecting miRNA-569 nucleic acid using a hairpin DNA composition having nucleotide sequences according to a second embodiment.

FIGS. 3 and 4 show results obtained by performing polyacrylamide gel electrophoresis. After amplifying DNA or miRNA, the products can be separated on a gel, and their molecular weights (sizes) and presence or absence can be confirmed.

In FIG. 3, H1, H2, and H3 denote hairpin DNA1, hairpin DNA2, and hairpin DNA3 corresponding to SEQ ID NOS: 2, 3, and 4, respectively, and in FIG. 4, H1, H2, and H3 denote hairpin DNA1, hairpin DNA2, and hairpin DNA3 corresponding to SEQ ID NOS: 5, 6, and 7, respectively.

In FIGS. 3 and 4, the "H1" lane shows the case where only hairpin DNA1 is present, the "H2" lane shows the case where only hairpin DNA2 is present, and the "H3" lane shows the case where only hairpin DNA3 is present. The "H1 H2 H3" lane indicates the case where hairpin DNA1, hairpin DNA2, and hairpin DNA3 are mixed together.

The "H1 H2 H3 mir 569" lane shows the result of generating a Y-shaped DNA structure by binding the target nucleic acid miRNA-569 to hairpin DNA1, hairpin DNA2, and hairpin DNA3 as in the present invention. As such, due to a high-molecular-weight complex such as the Y-shaped DNA structure, the "H1 H2 H3 mir 569" lane in each of FIGS. 3 and 4 exhibits the greatest emission of fluorescence signal due to the Y-shaped DNA structure, thereby confirming that detection of miRNA-569 was achieved.

In particular, in the results of FIG. 4 obtained using hairpin DNA1, hairpin DNA2, and hairpin DNA3 corresponding to SEQ ID NOS: 5, 6, and 7, the fluorescence signal was greater than in the results obtained using hairpin DNA1, hairpin DNA2, and hairpin DNA3 corresponding to SEQ ID NOS: 2, 3, and 4, indicating that, in the case of hairpin DNA1, hairpin DNA2, and hairpin DNA3 corresponding to SEQ ID NOS: 5, 6, and 7, binding with miRNA-569 was more highly amplified.

What is claimed is:

1. A DNA composition for detecting a target nucleic acid, comprising:
a single-stranded hairpin DNA1 comprising a nucleotide sequence
capable of complementary binding to the target nucleic acid, the single-stranded hairpin DNA1 having a stem portion in which portions of opposite ends thereof are complementarily bound to each other, and an unhybridized loop portion in the form of a loop;a single-stranded hairpin DNA2 comprising a nucleotide sequence
capable of complementary binding to the hairpin DNA1, the single-stranded hairpin DNA2 having a stem portion in which portions of opposite ends thereof are complementarily bound to each other, and an unhybridized loop portion in the form of a loop; and a hairpin DNA3 comprising a nucleotide sequence capable of
complementary binding to the hairpin DNA2, the hairpin DNA3 having a stem portion in which portions of opposite ends thereof are complementarily bound to each other, and an unhybridized loop portion in the form of a loop, the hairpin DNA3 having a fluorophore at one end thereof and a quencher, which is a fluorescence quencher, at an opposite end thereof.

2. The DNA composition of claim 1, wherein the hairpin DNA1 hybridizes with the target nucleic acid such that the stem portion of the hairpin DNA1 is opened, the hairpin DNA2 then hybridizes with the hairpin DNA1 such that the stem portion of the hairpin DNA2 is opened, the hairpin DNA3 then hybridizes with
the hairpin DNA2 such that the stem portion of the hairpin DNA3 is opened, and
another portion of the hairpin DNA3 displaces the target nucleic acid bound to the hairpin DNA1 and hybridizes with the hairpin DNA1 at that portion to form a Y- shaped DNA structure.

3. The DNA composition of claim 2, wherein, in the Y-shaped DNA structure, a fluorescence signal is emitted from the fluorophore provided at one end of the opened hairpin DNA3, thereby detecting the target nucleic acid.

4. The DNA composition of claim 1, wherein the target nucleic acid is
microRNA-569(hereinafter referred to as "miRNA-569"), having the nucleotide sequence of SEQ ID NO: 1.

5. The DNA composition of claim 4, wherein the hairpin DNA1 has the nucleotide sequence of SEQ ID NO: 2, the hairpin DNA2 has the nucleotide
sequence of SEQ ID NO: 3, and the hairpin DNA3 has the nucleotide sequence of SEQ ID NO: 4.

6. The DNA composition of claim 4, wherein the hairpin DNA1 has the nucleotide sequence of SEQ ID NO: 5, the hairpin DNA2 has the nucleotide
sequence of SEQ ID NO: 6, and the hairpin DNA3 has the nucleotide sequence of SEQ ID NO: 7.

7. A target nucleic acid detection sensor comprising the DNA composition for detecting a target nucleic acid of claim 1.

8. A method for detecting a target nucleic acid using the DNA composition for detecting a target nucleic acid of claim 1.

9. The method of claim 8, wherein the method for detecting the target
nucleic acid comprises:
(a) hybridizing the hairpin DNA1 with the target nucleic acid such that the stem portion of the hairpin DNA1 is opened;
(b) hybridizing the hairpin DNA2 with the hairpin DNA1 such that the stem portion of the hairpin DNA2 is opened;
(c) hybridizing the hairpin DNA3 with the hairpin DNA2 such that the stem portion of the hairpin DNA3 is opened; and
(d) displacing, by another portion of the hairpin DNA3, the target nucleic acid bound to the hairpin DNA1 and complementarily binding, at that portion, to the hairpin DNA1 to form a Y-shaped DNA structure,
wherein the method is a method for detecting miRNA-569 nucleic acid using the DNA composition.

10. The method of claim 9, wherein, in the Y-shaped DNA structure, a fluorescence signal is emitted from the fluorophore provided at one end of the
opened hairpin DNA3, thereby detecting the target nucleic acid.

11. The method of claim 8, wherein the target nucleic acid is microRNA- 569(hereinafter referred to as "miRNA-569"), having the nucleotide sequence of SEQ ID NO: 1.

12. The method of claim 11, wherein the hairpin DNA1 has the nucleotide sequence of SEQ ID NO: 2, the hairpin DNA2 has the nucleotide sequence of SEQ ID NO: 3, and the hairpin DNA3 has the nucleotide sequence of SEQ ID NO:4.

13. The method of claim 11, wherein the hairpin DNA1 has the nucleotide sequence of SEQ ID NO: 5, the hairpin DNA2 has the nucleotide sequence of SEQ ID NO: 6, and the hairpin DNA3 has the nucleotide sequence of SEQ ID NO:7.
